(19) United States
(12) Patent Application Publication
(10) Pub. No.: US 2024/0118122 A1
(43) Pub. Date: Apr. 11, 2024

(54) CAPACITIVE SENSING FOR BLOOD CHARACTERIZATION

(71) Applicant: UNIVERSITY OF WASHINGTON, Seattle, WA (US)

(72) Inventors: Praveen Kaliappan Sekar, Seattle, WA (US); Dayong Gao, Seattle, WA (US); Jae-Hyun Chung, Seattle, WA (US); Yanyun Wu, Seattle, WA (US)

(73) Assignee: UNIVERSITY OF WASHINGTON, Seattle, WA (US)

(21) Appl. No.: 18/263,573

(22) PCT Filed: Feb. 1, 2022
(86) PCT No.: PCT/US2022/014683

§ 371 Date: Jul. 31, 2023

Related U.S. Application Data
(60) Provisional application No. 63/144,389, filed on Feb. 1, 2021.

(51) Int. Cl. G01F 23/263; G01N 33/487; G01N 33/49

(52) U.S. Cl. CPC G01F 23/266; G01F 23/268; G01N 33/48771; G01N 33/4905; G01N 2800/224

(57) ABSTRACT

A capacitive sensor system configured to measure capacitance, including a sample volume, a sample capacitive sensor configured to measure the capacitance of the sample volume without physical contact between the sample capacitive sensor and the sample volume, a control capacitive sensor, a differential sensing subsystem configured to measure a control sensor volume using the control capacitive sensor, and electrical circuitry connected to both the control capacitive sensor and the sample capacitive sensor.

### CROSS-REFERENCE TO RELATED APPLICATION

This application claims the benefit of U.S. Provisional Application 63/144,389 filed on Feb. 1, 2021, the disclosure of which is hereby expressly incorporated by reference in its entirety.

### BACKGROUND

The primary method by which a body responds to an injury or trauma is the formation of a thrombus to stop bleeding. Hemostasis is a highly complex process involving both non-cellular plasma proteins and cellular components in the blood. Initially, at the site of the wound, platelets quickly aggregate to form a temporary plug to seal the blood vessel. The clotting factors in the plasma take part in a cascade of chemical reactions that culminate in the conversion of soluble fibrinogen to a network of insoluble fibrin mesh, encompassing red blood cells and the aggregated platelets. Platelets in the clot, exert contractile force pulling the fibrin strands, bringing other platelets and red blood cells together, thereby tightening the clot and drawing the vessel walls together. The strength and stability of a hemostatic clot is dependent on the quantity (concentration) and quality (function) of both coagulation factors and platelets. Patients with platelet and/or coagulation factor disorders, and/or in trauma, and/or under the administration of antiplatelet or anticoagulant drugs, have impaired clotting ability, thereby leading to a profuse loss of blood.

Evaluating the hemostatic ability of a patient in a timely manner is critical for making decisions on transfusions and other therapeutic management. Currently available conventional coagulation tests such as activated partial thromboplastin time(aPPT) or prothrombin time (PT) are performed on blood plasma (devoid of blood cells), which require transport of samples to a laboratory with trained personnel and a step of centrifugation, significantly increasing the turnaround time, typically more than 1 to 2 hrs. Also, they focus only on the coagulation factor aspect of the clot providing no concurrent information on the platelets. A few point-of-care coagulation devices such as CoaguChek. (Roche) and i-STAT (Abbot) are available; however, they exhibit poor performance in terms of repeatability and are primarily limited to monitoring only a specific condition like for example warfarin anticoagulation therapy. Thromboelastography (TEG) and rotational thromboelastometry (ROTEM) are two viscoelastic whole blood-based assays, which are increasingly used for treatment and diagnosis of bleeding or clotting disorders, for trauma management and surgery support. They analyze different hemostatic aspects like providing insights on both the coagulation factors and platelet function, to some extent, however, they still require trained professional to perform and interpret the results and are frequently prone to sampling and processing errors. In addition to TEG and ROTEM, there are also laboratory specialized platelet functions assays such as platelet aggregation that can be used to diagnosis platelet dysfunction. These assays can only be performed locally (as relatively fresh platelets are needed) and in specialized laboratory by trained professional for testing and interpretation.

As such, methods and devices for rapid and accurate clinical assessments of hemostasis are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Overall, the presented CPC capacitance sensor is a biomedical device for convenient non-contact, whole-blood based comprehensive hemostasis evaluation. The presented technology generally relates to systems consisting of one or more capacitive sensors, including carbon nanotube-paper composite sensors, for measuring one or more blood characteristics such as measuring and monitoring coagulation ability based on plasma-coagulation factors and platelets, and/or performing platelet counts and/or hematocrit measurements in whole blood, and associated methods and protocols.

In one aspect, A capacitive sensor system configured to measure capacitance, the system including a sample volume, a sample capacitive sensor configured to measure the capacitance of the sample volume without physical contact between the sample capacitive sensor and the sample volume, a control capacitive sensor, a differential sensing subsystem configured to measure a control sensor volume using the control capacitive sensor, and electrical circuitry connected to both the control capacitive sensor and the sample capacitive sensor is disclosed.

In another aspect, a method of assessing one or more blood conditions using the sensor described herein, the method including obtaining a whole blood sample, measuring the capacitance of the whole blood sample with a sample capacitance sensor that is not in contact with the whole blood sample, comparing the capacitance of the whole blood sample to the capacitance of a control volume, and assessing three or more biomarkers, wherein the three or more biomarkers include coagulation function, platelet count and quality, and red blood cell concentration is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example carbon nanotube-paper composite capacitive sensor, in accordance with the present technology;

FIG. 1B is an example sensor, in accordance with the present technology;

FIG. 1C is an example capacitive sensor system, in accordance with the present technology;

FIG. 1D is an example capacitive sensor system, in accordance with the present technology;

FIG. 2 is a graph of the capacitance change with sample addition (SC) for various operating frequencies, in accordance with the present technology;

FIGS. 3A-3E are graphs of system biomarkers from a coagulation measurement and their relationship with specific characteristics of whole blood, in accordance with the present technology.

FIGS. 4A-4C are graphs of comparison with Thromboelastography (TEG), in accordance with the present technology FIG. 5A (Suppl. FIG. 1A) is a photo photograph of an example capacitive sensing device, in accordance with the present technology; and

FIGS. 5A-5F are graphs showing multiparameter hemostasis assessment for hemophilia and thrombocytopenia conditions, in accordance with the present technology;

FIG. 6 is a SEM image of an example Carbon nanotube composite, in accordance with the present technology;

FIGS. 7A-7D are example capacitance sensor noise level, calibration, and characterization in accordance with the present technology;

FIG. 8A is an example observation of sedimentation at different orbital rotational speed, in accordance with the present technology;

FIG. 8B is a graph of the time course variation of capacitance for healthy whole blood subjected to orbital shaking, in accordance with the present technology;

FIG. 9A is a graph of a capacitance signal after orbital shaking, in accordance with the present technology;

FIG. 9B is example clotting after orbital shaking, in accordance with the present technology;

FIGS. 10A-10H are graphs of sensing parameters, in accordance with the present technology;

FIGS. 11A-11C are graphs of stability assessments by an example sensor in accordance with the present technology; and

FIGS. 12A-12C are graphs of example distribution sensing parameter for health volunteers; in accordance with the present technology.

### DETAILED DESCRIPTION

While illustrative embodiments have been illustrated and described, it will be appreciated that various changes can be made therein without departing from the spirit and scope of the invention.

Hemostasis is a complex physiological cascade in which coagulation factors, platelets and erythrocytes all play prominent roles. Rapid and accurate assessment of these key parameters is imperative in various clinical settings to diagnose, treat, and monitor patients with impaired hemostasis. Over 15 million people worldwide receive oral anticoagulant therapy to prevent and treat life-threatening thromboembolic events, such as deep venous thrombosis, pulmonary embolism, myocardial infarction and stroke. Despite their effectiveness in lowering the risk of acute thrombosis, effective anticoagulation management in these patients remains challenging because of the narrow therapeutic window between either increased risk of thromboembolic events (inadequate treatment) or higher probability of dangerous hemorrhage and life-threatening organ failure (over-administration), which is influenced by numerous food and drug interactions, underlying comorbidities and variability in dose response among patients. Therefore, patients require frequent laboratory testing of blood clotting status to ensure appropriate selection of anticoagulant agent and dosing, which imposes a staggering service load on health care providers. Together, these factors underscore the dire unmet need for inexpensive routine home-monitoring of oral anticoagulation status to advance the quality of care in patients receiving oral anticoagulant therapy and improve utilization of health care resources. Traditional laboratory-based clotting assays and devices are limited to not only time-consuming and expensive devices, but also provide insufficient information due to partial analysis of various individual facetted clotting elements, such as coagulation function (activated partial thromboplastin time—aPTT, prothrombin time—PT, thrombin time—TT), fibrinogen, platelet function (platelet functional analyzer and aggregometry) or blood cellularity (hematology analyzers). Evolving whole blood-based viscoelastic assays such as thromboelastography (TEG) also lack sensitivity and specificity to platelet counts and platelet dysfunction, and can often provide an inaccurate assessment of hemostatic status in the settings of abnormal hematocrit. As a result, each current assay or testing technology has provided an incomplete assessment of bleeding and thrombosis status and risks. This has also stymied the fields of clinical and experimental hematology given the interdependence among the clotting elements.

Over the years, electrical permittivity sensors have been extensively developed to characterize various blood physiological properties that are of relevance for numerous medical applications including hemostasis. The permittivity change of whole blood in the MHz range was from the accumulation of charge carriers at the interface between erythrocyte membrane and its cytoplasm, and this was found to change significantly during cellular aggregation, formation of fibrin mesh networks encapsulating the aggregates and retraction of the clot from the contractile force exerted by activated platelets. So far, two systems that measure blood permittivity using an impedance technique have demonstrated a good sensitivity to both coagulation and platelet function. One study has reported an independent evaluation of hematocrit level without clotting. These studies confirmed that measuring blood permittivity can provide a sensitive analysis of both cellular and non-cellular blood components that play a key role in hemostasis. However, these impedance-based devices were not able to offer concurrent information on all the key clotting elements from a single assay, and also have limitations in being translated into clinical practice. The physical electrical contact with the blood samples irreversibly contaminates the sensing surface from corrosive elements in the blood (such as proteins and ions). Also, these single-use disposable sensors require precious metal (such as Au or Pt) electrodes to reduce oxidation and electrostatic contamination, coupled with a highly specialized impedance analyzers, making them expensive to use in clinical settings. Moreover, the physical electrical contact can also interfere with the natural progression of clotting process in the blood samples. Addressing the challenges in electrical sensor resolution and sensitivity can lead to insights into key components of hemostasis that arise from studying blood permittivity.

Rapid and accurate clinical assessment of hemostasis is essential for managing patients who undergo invasive procedures, experience hemorrhages, or receive antithrombotic therapies. Hemostasis encompasses an ensemble of interactions between the cellular and non-cellular blood components, but current devices assess only partial aspects of this complex process. Described herein is the development of a new approach to simultaneously evaluate coagulation function, platelet count or function, and hematocrit using a carbon nanotube-paper composite (CPC) capacitance sensor.

In one aspect, a capacitive sensor system configured to measure capacitance, the system comprising a sample volume, a sample capacitive sensor configured to measure the capacitance of the sample volume without physical contact between the sample capacitive sensor and the sample volume, a control capacitive sensor, a differential sensing subsystem configured to measure a control sensor volume using the control capacitive sensor, and electrical circuitry connected to both the control capacitive sensor and the sample capacitive sensor is disclosed. In some embodiments, a capacitance-based approach for blood permittivity measurement using a carbon nanotube-paper composite (CPC) capacitance sensor is disclosed. A major advancement of the proposed approach is that the capacitance signal obtained from a single measurement can provide three parameters with distinctive sensitivity towards coagulation function, platelet count and/or function, and hematocrit. The CPC sensor exhibits high fringing field to detect permittivity changes in a blood sample placed in a glass vial on top of the sensor, eliminating the need for physical electrical contact. The feasibility and sensitivity of this approach is demonstrated herein using clinically relevant human blood samples. The results were compared against a conventional TEG analyzer to validate the effectiveness of the sensor. To demonstrate potential clinical usefulness, the reduced hemostatic ability was evaluated in terms of coagulation function, platelets and hematocrit for hemophilia and thrombocytopenia conditions.

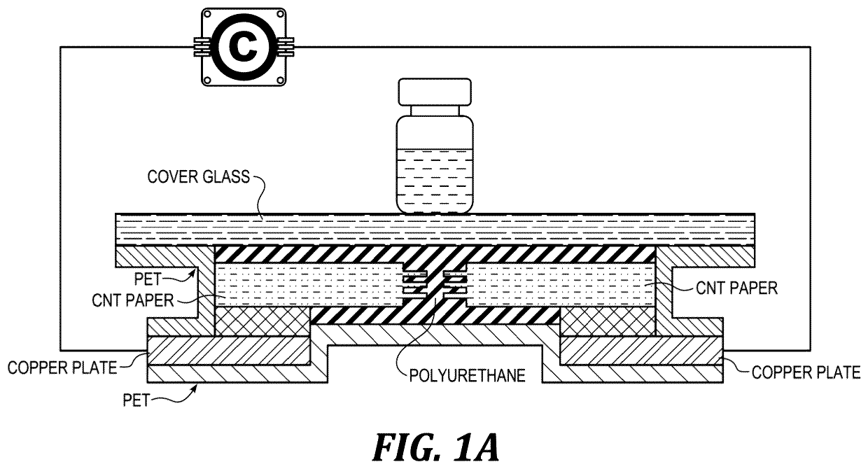

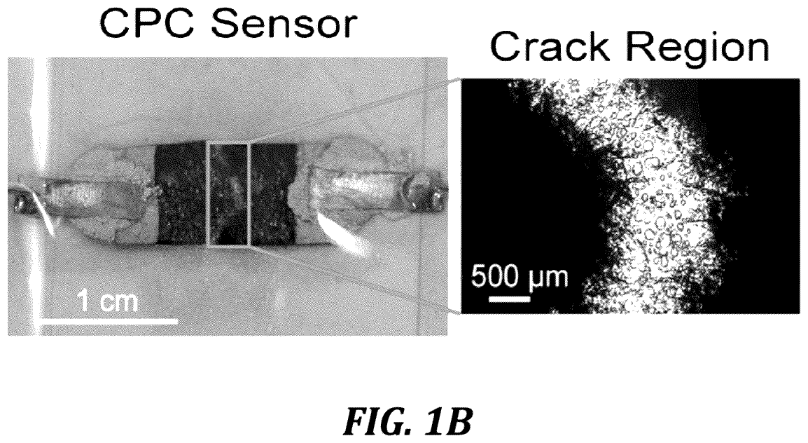

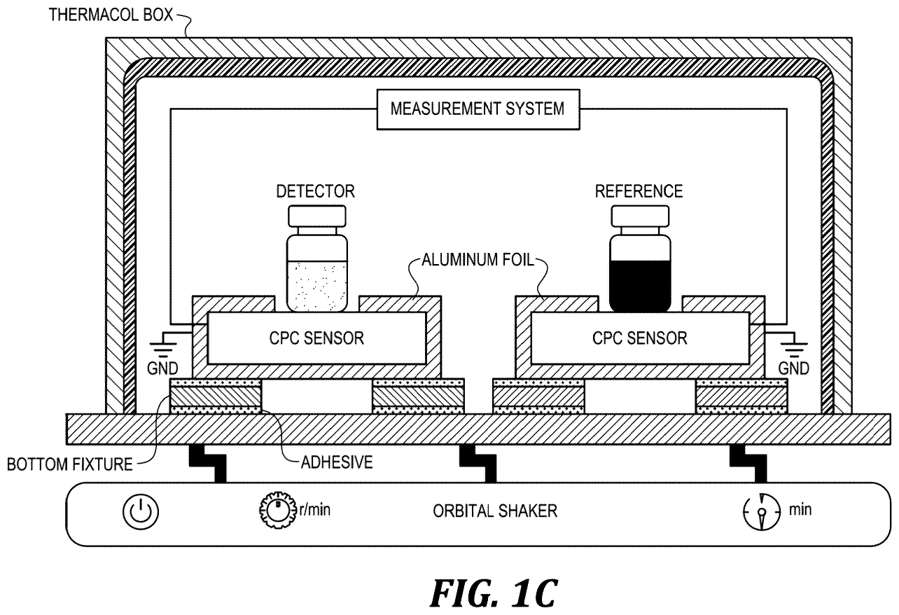

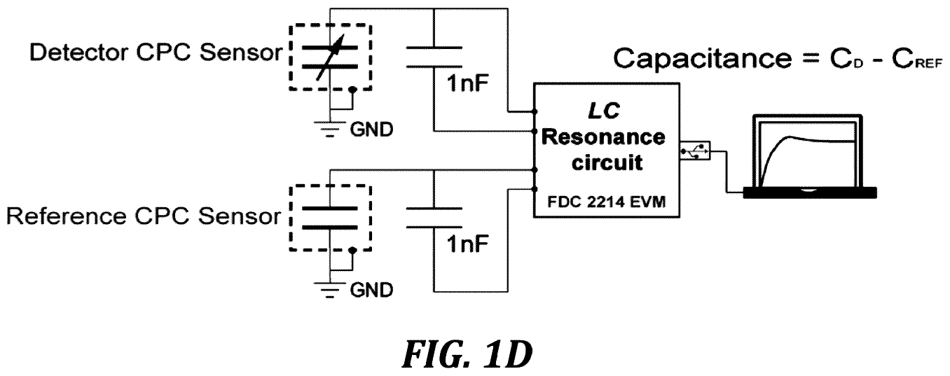

FIG. 1A is an example carbon nanotube-paper composite capacitive sensor, in accordance with the present technology. Illustrated is a schematic layout of the experimental setup and method for hemostasis assessment. In some embodiments, the system includes a carbon nanotube (CNT) paper sandwiched between polyurethane. In some embodiments, the sensor system is on PET substrate. In some embodiments, the sensor is coated with silver epoxy. In some embodiments, a cover glass is placed on top of the sensor system.

In some embodiments, the overall system includes (i) two or more capacitive sensors including carbon nanotube-paper composite sensors (ii) electrical circuitry for differential-capacitance measurement, (iii) an orbital oscillator or a shaker with/without temperature control, (iv) two or more glass or plastic or any whole blood compatible vials to hold the sample (SUT), placed on top of the sensor, and (v) fixtures to host the capacitive sensors and vials. In some embodiments, the system consists of at least two such sensors (i) one control or reference sensor (Cntrl) and (ii) one or more sample (or detector) sensors. In operation, any changes in the electric field between the electrodes of each of the sensor, caused by changes in dielectric property due to a change in medium and/or change within a medium due to physiological change such as coagulation, will cause a measurable change in the capacitance of the sensor. The measured capacitance is the differential measurement between the sample measurement sensor and the Cntrl sensor.

In some embodiments, the capacitive sensing system includes electrical circuitry that connects a control capacitive sensor and a sample capacitive sensor. In some embodiments, the electrical circuitry uses pulse-counting or resonance between 10 kHz and 10 MHz. In some embodiments, CPC capacitance response to blood clotting at 1.3 MHz provided three sensing parameters with distinctive sensitivities towards multiple clotting elements. In an example, whole blood-based hemostasis assessments were conducted to demonstrate the potential utility of the developed sensor for various hemostatic conditions, including pathological conditions, such as hemophilia and thrombocytopenia. Results showed good agreements when compared to a conventional thromboelastography. In some embodiments, the electrical circuitry for differential capacitive measurement is configured to measure the sample capacitive sensor capacitance from the change and/or shift in resonant frequency of an inbuilt tank circuit, in parallel to the sample capacitive sensor. In some embodiments, the electrical circuitry is configured to calibrate temperature change.

In some embodiments, the capacitive sensor, the control sensor, or both sensors are carbon-nanotube cellulose paper composite sensors. In some embodiments, a capacitive sensor is fabricated by controlled tensile fracture of carbon nanotube infused paper (cellulose), and vacuum sealed to a cover glass on the sensing side and a polyester film on the other. In some embodiments, cover glass is supported at the ends to prevent pressure on the sensor. In some embodiments, the vials containing the sample under test are positioned on top of the capacitive sensors, on the cover-glass side.

FIG. 1B is an example sensor, in accordance with the present technology. The close up illustrates the crack region of the sensor and is zoomed in at 4× magnification. In some embodiments, such as the one illustrated in FIG. 1D, untangled cellulose fibers form numerous conductive cantilevers exhibiting high fringing field for capacitance measurements.

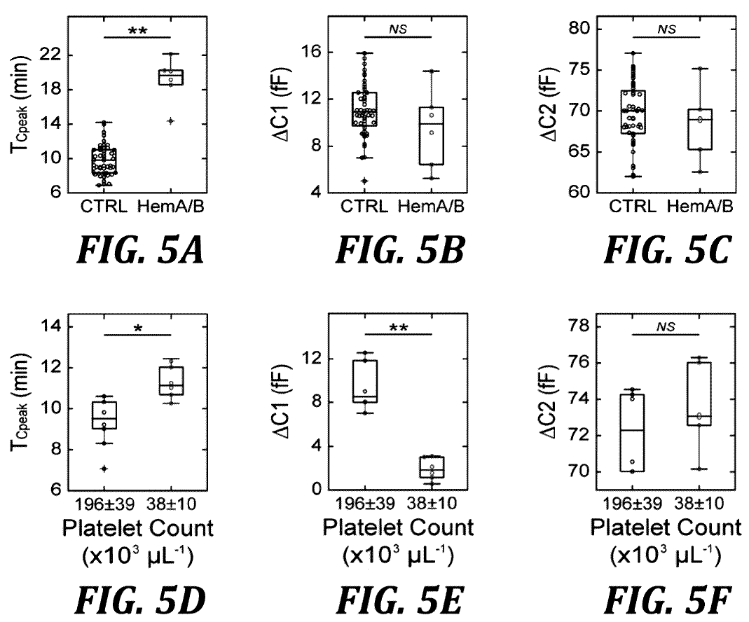

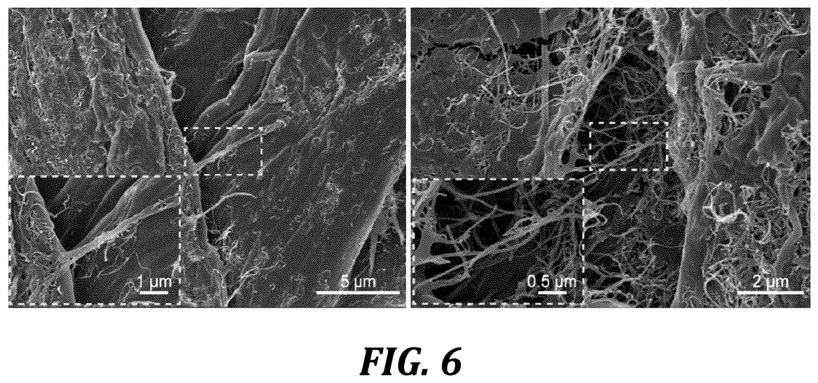

Carbon nanotube-paper composite (CPC) film made of multiwall carbon nanotubes (MWCNTs) and cellulose fibers may be prepared and fabricated as a capacitance sensor as shown in FIGS. 1A and 1B. In some embodiments, bleached softwood pulp pre-adsorbed with cationic polyacrylamide (CPAM, Percol 3035; BASF, RP, DE) was mixed with a dispersion of sodium dodecyl sulfate (Sigma Aldrich, MO, U.S.A.) and MWCNTs (Cheap Tubes Inc., VT, U.S.A.). The resulting aqueous suspension was subsequently filtered, pressed and dried to form electrically conductive sheets of 60 g m⁻² containing 10% (w/w)-MWCNTs. In some embodiments, the sheets are cut into 5 mm wide strips with 20 mm in length. In some embodiments, silver epoxy (MG Chemicals, BC, CA) is patterned at both the ends to provide electrical contact. The SEM images of the CPC surface showed that the cellulose fibers were uniformly coated with the MWCNTs, as seen in FIGS. 5A-5B as well as FIG. 6. In some embodiments, controlled water printing and stretching techniques are used to fabricate the CPC capacitance sensors with a V-shaped crack, but may also have a straight-line crack. The V-shaped crack demonstrates a higher sensitivity in comparison to a straight-line crack due to larger surface area. Water may be printed on the CPC specimens using a noncontact liquid bridge printing method. Then, stretching may be applied using a tensile test stage to fabricate the sensor electrodes with a gap of 1 mm. The average capacitance for six sensors from the same batch was found to be 314±12 fF (3.8%; CV). A randomly selected pair of sensors were used for all measurements described herein. Standard cold soldering through copper plates was utilized for external wiring. The fractured CPC may be coated with polyurethane and vacuum-sealed on a cover glass (0.17 mm thickness, Thermo Scientific, MA, U.S.A). A polyester film may be used to protect the sensor from oxidation and environmental exposure. In some embodiments, the sample capacitive sensor and/or the control capacitive sensor is hermetically sealed to avoid the effect of humidity change and oxidation of sensor surfaces.

FIG. 1C is an example capacitive sensor system, in accordance with the present technology. An example schematic of the CPC capacitive sensor is illustrated, not drawn to scale. In some embodiments, the capacitive sensor system includes vials to hold samples. In some embodiments, human whole blood is placed into the vial and on top of the CPC sensor and the Cntrl sensor. In some embodiments, the system includes a cover glass. In some embodiments, the cover glass is supported at the ends through plastic fixtures to avoid stress from sample weight on the carbon nanotube coated cellulose fibers.

In some embodiments, the electrical circuitry for capacitance measurement consists of at least two channels for the Cntrl and sample measurement sensor, excited at or in the range of 10 kHz to 10 MHz. In some embodiments, the sample measurement sensor is excited at 1.3 MHz. In some embodiments, the system includes vials to hold both the sample volume and the control volume. In some embodiments, the vials are made of anything compatible with whole blood material. In some embodiments, the vials are made of glass. In other embodiments, the vials are made of plastic. In some embodiments, the capacitive sensor system includes one or more housings configured to position the sample capacitive sensor in relation to the sample volume and position the control capacitive sensor in relation to the control volume. In some embodiments, the vials are held in position using cartridges to control the position on the sensors. The vials are positioned right above the gap between the capacitive sensor electrodes, to maximize the electric field through the sample. In some embodiments, the one or more housings are fabricated using 3D printing, injection molding, or milling technology. In some embodiments, the sample volume is not within a plane formed by electrodes of the sample capacitive sensor. In some embodiments, the sample volume is, at its nearest point, set apart from the place formed by the electrodes by a distance of 0.1 mm to 10 mm. In some embodiments, the entire system is placed on an orbital oscillator or shaker mechanism and covered using a container to prevent the effect of temperature on samples and electrical measurement system. In some embodiments, the container is a Thermacol or Styrofoam container.

In some embodiments, the control volume is selected from a physiological buffer or other liquid sample with uniform dielectric constant. In some embodiments, the control volume is DI water. In some embodiments, the control volume is used to calibrate the capacitive sensor system related to a characteristic selected from volume, dielectric constant, liquid temperature, or combinations thereof.

In some embodiments, the experimental setup used for capacitance measurement consists of two CPC capacitance sensors, FDC 2214 capacitance evaluation module (Texas Instruments, TX, U.S.A), glass vials (Agilent Technologies, CA, U.S.A), 3D printed plastic fixtures and a container, such as a Styrofoam box, assembled on the orbital shaker (KJ-201 BD, WINCOM, Jiangsu, CHN) as shown in FIG. 1C.

FIG. 1D is an example capacitive sensor system, in accordance with the present technology. Illustrated is a functional block diagram of the measurement system. In some embodiments, a laptop with data acquisition and processing software, may be used as both a power source and data logger. In an example, an FDC 2214 evaluation module was composed of an inductance-capacitance (L-C) circuit to measure capacitance changes as shifts in resonant frequency of the L-C circuit. A 1 nF ceramic capacitor was integrated parallel to the L-C circuit to achieve an excitation frequency of 1.3 MHz.

In some embodiments, the one or more housings are plastic fixtures. In some embodiments, a blood sample is placed into two glass vials and precisely positioned using plastic fixtures on top of the crack region in the sensor to ensure maximum fringing field for high sensitivity. Fixtures also minimize the variability in sample position between measurements. Bottom fixtures may support the sample weight eliminating any mechanical pressure on the MWCNT-coated cellulose fibers. To reduce the parasitic capacitance, aluminum (Al) foil may be used to shield the sensors and the inner surface of the Styrofoam box.

In some embodiments, the sample volume is whole blood. In some embodiments, the sample volume is whole human blood, but it may be the blood of any animal that has a hemostasis and clotting system. In some embodiments, the blood sample is between 1 ml and 500 ml. In some embodiments, the blood sample is a finger prick, such as the amount needed for iron or diabetes testing. In some embodiments, for whole blood coagulation/hemostasis measurements, citrated human whole blood samples are added to both the Cntrl and sample measurement vials. In operation, the sample measurement vials may be prefilled with agonist solutions like thrombin or calcium chloride to activate a clot formation, upon addition of sample. After the addition of sample to both the vials, the orbital oscillator or shaker mechanism is turned on. The oscillator/shaker system prevents RBC rouleaux formation and sedimentation in the sample, making the measurement specific to the clotting physiology changes in the activated sample. After each measurement, only the vials are disposed, minimizing the need for recalibration of the sensor. A differential capacitance measurement minimizes the effect of noise from environmental fluctuations such as temperature and/or mechanical and/or electrical.

Such electrical readout offers superior performance characteristics such as better sensitivity, accuracy, repeatability, reliability, and portability over an optical measurement system, commonly used in currently established techniques. The superior performance characteristics of the device enables successful capture of the clotting dynamics during hemostasis and provide specific biomarkers dependent on plasma-coagulation factors and platelets. In addition to evaluating the clotting ability, the system can also provide a specific measurement for hematocrit and platelet counts.

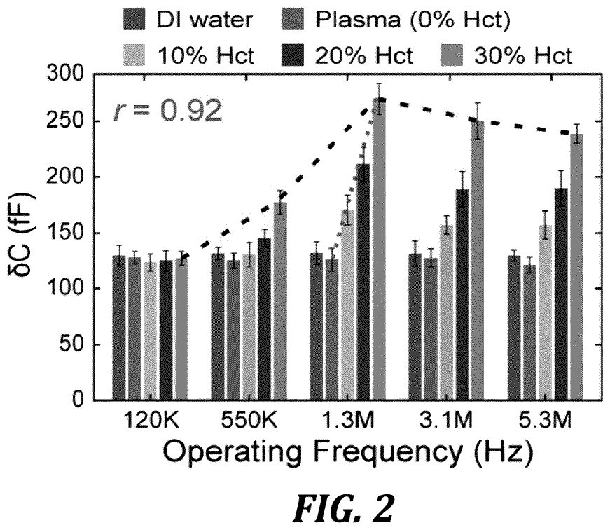

FIG. 2 is a graph of the capacitance change with sample addition (δC) for various operating frequencies, in accordance with the present technology. Peak δC was observed at 1.3 MHz for all the hematocrit measurements. Shown is a total of three samples with single replicates. Bars represent mean values. Margins of error are illustrated in each measurement. At 1.3 MHz, δC linearly increases with hematocrit. As shown, Pearson's r=0.92.

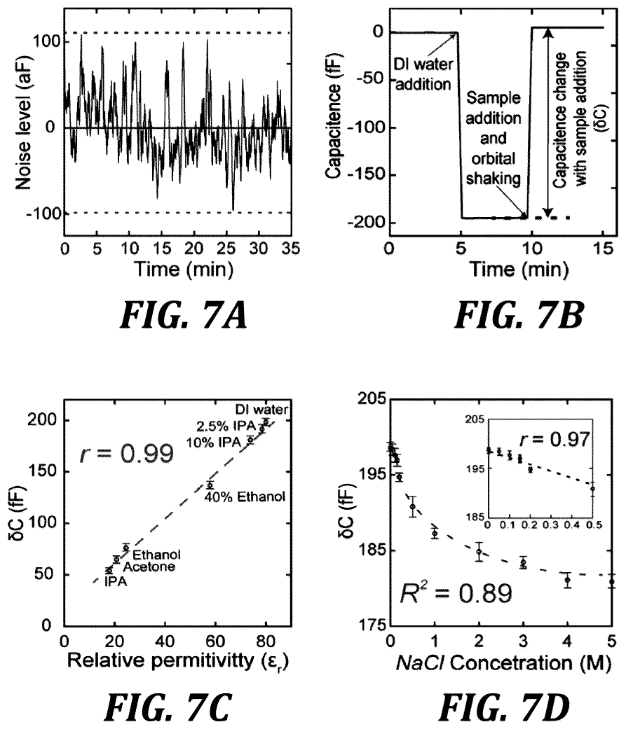

In operation, glass vials may be placed on top of the sensors and initial capacitance may be measured for a length of time. In some embodiments, the length of time is 5 minutes. In some embodiments, the control sample is DI water. In one example, 500 μL, of DI water was added to the reference sensor and allowed to equilibrate for 5 min. FIGS. 7A-7D demonstrate the measurement protocol used in the experiments shown in FIG. 2. Subsequently, 500 μL, of the testing sample was added to the detector sensor and 80-rpm shaking was applied. Measurement was performed for an additional 5 min. Capacitance change from sample addition (δC) was evaluated as the difference between the averaged capacitance before (7-10 minutes, as shown in FIG. 7B) and after the addition of testing sample (12-15 minutes, as shown in FIG. 7B).

### Example

In some embodiments, the system is configured to evaluate the over-all clotting ability of a subject by delineating plasma-mediated and platelet-mediated hemostasis events, providing specific biomarkers or measurement for coagulation factors, platelets, clotting formation, or other hemostasis or clotting measurements. Clotting measurements were conducted using unmodulated blood and blood sample modulated for a predefined platelet count or platelet function or hematocrit, which was generally referred to as blood sample herein. In some embodiments, the subject is an animal with a hemostasis and clotting system, but the subject may also be a human.

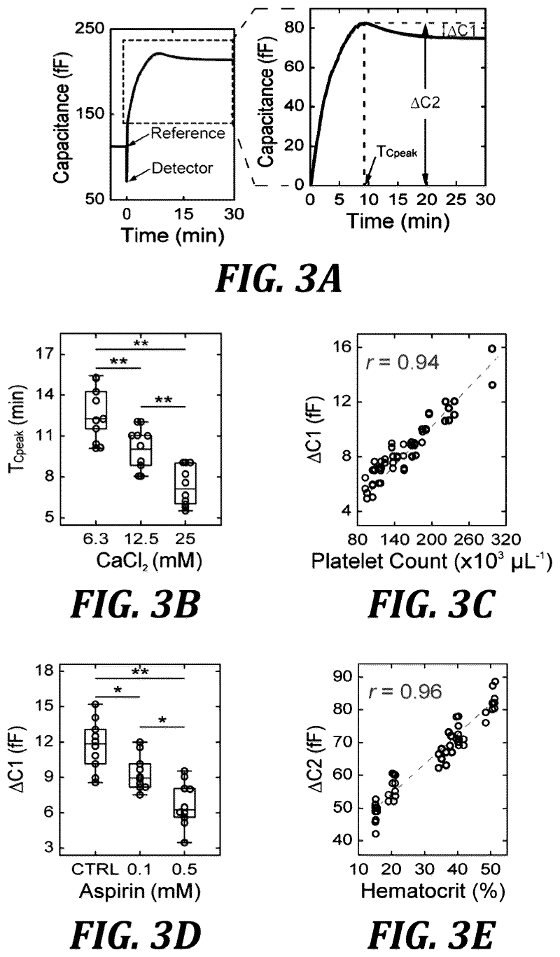

FIGS. 3A-3E are graphs of system biomarkers from a coagulation measurement and their relationship with specific characteristics of whole blood, in accordance with the present technology. For a clotting assay as shown in FIG. 3A, a baseline capacitance was measured for 5 min with the glass vials placed on the sensors. On the left, is a representative overall capacitance signal for a blood sample activated using CaCl₂) (12.5 mM). On the right, is a zoomed-in view of the region of interest with the sensing parameters, T_{Cpeak}, ΔC1, and ΔC2 for analyses. In operation, 325 μL of the blood sample is added to the reference sensor. Immediately, 300 μL of the same blood is added to the detector sensor and 80-rpm orbital shaking is applied. The glass vial on the detector sensor was prefilled with 25 μL of 162.5 mM CaCl₂ solution (final Ca²⁺ concentration in whole blood: 12.5 mM) for recalcification of the blood sample. Differential capacitance was measured for an additional 30 min after activation. All measurements were conducted at room temperature (23° C.).

In some embodiments, the capacitive sensing system is able to concurrently measure the clotting ability, hematocrit, and platelet count of a whole blood sample. In some embodiments, the system is configured to detect both quantitative platelet defects (i.e. too many, or too few platelets), and qualitative platelet defects (i.e. functionality defects). To examine the ability of the sensor to simultaneously assess coagulation function, platelets and hematocrit, the blood samples from healthy donors were modulated for a predefined clotting activation level (Ca²⁺ concentration), platelet count or function, or hematocrit. From the measured capacitance, three parameters namely T_{Cpeak}, ΔC1 and ΔC2 were chosen for analysis as shown in FIG. 3A. It was found that T_{Cpeak} decreased with increasing CaCl₂), which indicated a trend in T_{Cpeak} and coagulation time (F(2,27)=27.16, p<0.00001; ANOVA). FIG. 3B illustrates the increasing CaCl₂) concentrations of 6.3, 12.5 and 25 mM. T_{Cpeak} decreases with increasing concentration of activating CaCl₂) (mM). For the remaining studies, 12.5 mM CaCl₂) was used to activate clotting in the samples.

When blood samples were tested with a predefined platelet count (100, 120, 140, 160 and 180×10³ platelets μl⁻¹), it was found that ΔC1 linearly increased with increasing platelet count (r=0.94, p<0.00001; Pearson's correlation) as shown in FIG. 3C. FIG. 3C shows ΔC1 linearly increases with platelet count in the range 93-298×10³ platelets μl⁻¹. The Pearson's r=0.94.

In some embodiments, the capacitive sensor system is configured to evaluate or monitor the effects of antiplatelet and anticoagulant factors on hemostasis. In some embodiments, the antiplatelet or anticoagulant factor is a medication. In some embodiments, the medication is aspirin. From measurements using blood samples incubated with aspirin (0, 0.1, 0.51 mM), which inhibits platelet activation, it was found that ΔC1 dose-dependently decreased with increasing aspirin concentrations (F(2,27)=17.68, p<0.0001; ANOVA) as shown in FIG. 3D. FIG. 3D shows ΔC2 linearly increases with hematocrit in the range 15.1%-51.2%. The Pearson's r=0.96. In FIG. 3A-3D, 5 healthy donors with two replicate measurements are illustrated. Black circles are results from blood samples treated in vitro to modulate platelet count or platelet function or hematocrit, and blue circles are results from blood samples without any such treatments. All box plots with whiskers represent data distribution (Maximum, Third quartile, Median, First quartile, Minimum). p-values were calculated from one way ANOVA with Tukey's post-hoc. * p<0.05 and ** p<0.01.

In some embodiments, the system is configured to measure and count platelets and hematocrit. Together, the above two results illustrated in FIGS. 3C and 3D show that ΔC1 was sensitive to both platelet count and function. When blood samples were tested with a predefined hematocrit (15, 20, 40 and 50%), it was found that ΔC2 linearly increased with increasing hematocrit (r=0.96, p<0.00001; Pearson's correlation) as shown in FIG. 3E. Collectively, the above results showed that the sensor can offer a multiparameter assessment of hemostasis by providing concurrent information on coagulation function, platelet count or function, and hematocrit.

Capacitance was measured at a sampling rate of 33 Hz during clotting. A real-time average of 300 data points (approximately 9 sec) was performed to obtain a very low frequency envelop for capacitance change with the progression of clotting. For data analysis and presentation (graphs), the initial time (t=0) was defined as the time point when the blood sample was activated. T_{Cpeak} was the timepoint for maximum capacitance value after activation. ΔC1 was evaluated as the magnitude decrease in capacitance from peak to a steady state value. Steady state capacitance was the averaged value between 28-30 min after activation. ΔC2 was the maximum capacitance value after activation.

Fresh whole blood in standard 3.2% citrate tubes were purchased from Bloodworks Northwest Seattle, WA. Unless specified, all blood samples were from de-identified, healthy volunteers without previously known coagulation or platelet disorders, and had not received any antithrombotic medication in the two weeks prior to sample collection. Blood tubes were transported and stored in a Styrofoam box at room temperature and used within 4 hours from blood draw. CaCl₂) solution in phosphate buffered saline (PBS, pH 7.4; Sigma-Aldrich, MO, U.S.A) was prepared on the day of experiments. In some experiments, the blood samples were treated in vitro to modulate platelet count or platelet function or hematocrit to systematically study their effects on the sensing parameters (T_{Cpeak}, ΔC1 and ΔC2). Blood samples with a predefined platelet count or hematocrit were prepared from two-step centrifugation technique. First, citrated whole blood was centrifuged at 200 g for 10 min to pellet down the erythrocytes. Desired volume of the resultant supernatant, platelet-rich-plasma (PRP) was carefully removed. Subsequently, a part of the collected PRP was further centrifuged at 4000 g for 5 min to obtain platelet-poor-plasma (PPP). The erythrocyte pellet was then resuspended in the remaining PRP by gently inverting the tube to get a reconstituted hematocrit modulated sample. The reconstituted sample was then divided into portions and diluted with calculated volume of PRP and PPP to yield a blood sample with a predefined hematocrit (15, 20, 40 and 50%) or platelet count (100, 120, 140, 160 and 180×10³ platelets μl⁻¹).

Platelet counts in all hematocrit modulated samples were maintained constant as 165±27×10³ platelets μl⁻¹. Similarly, the hematocrit of all platelet count modulated samples was 40±3%. Platelet functional inhibition was done by incubating the samples with a predefined concentration of aspirin (0, 0.1, 0.51 mM). Aspirin (Sigma-Aldrich, MO, U.S.A) was dissolved in dimethyl sulfoxide (DMSO; Sigma-Aldrich, MO, U.S.A) and diluted with PBS to a desired concentration before adding to blood samples. In the aspirin study, appropriate amount of PBS buffer was added to the control samples to maintain approximal cell counts and hemodilution as the aspirin-treated samples. Platelet counts and hematocrit in whole blood, PRP and PPP were measured using a hematology analyzer (XP-300, Sysmex America OR, U.S.A) with an accuracy of 1.5% for hematocrit and ±6×10³ μl⁻¹ for platelet counts. In general, ethylenediaminetetraacetic acid (EDTA) is preferred for blood cellularity measurements to avoid hemodilution (Briggs, 2009). However, EDTA irreversibly chelates the free calcium in blood and permanently affects the progression of coagulation cascade and the ability of the blood cells to form aggregates and deform from retraction forces (Green et al., 2008). Therefore, blood counts were measured on blood samples anticoagulated with sodium citrate and reported with 10% hemodilution.

TEG measurements were performed by trained personnel at the Department of Laboratory Medicine, University of Washington using TEG 5000 thromboelastograph hemostasis analyzer (Haemonetics, U.S.A). R-time parameter is the time from the start of the measurement to the initial detection of clot formation (in min) and is indicative of the coagulation function. MA is a measure of clot strength as determined by the ability of platelets to bind via an α_{IIb}β₃ integrin as well as the elasticity of the fibrin mesh (Gottumukkala et al., 1999). The newly introduced sensing parameters, T_{Cpeak} and ΔC1, were compared with the equivalent TEG diagnostics parameters R-time and MA, respectively. TEG measurements were performed within 5 hours from blood draw. Blood samples were activated with 12.5 mM CaCl₂ (Citrated Native TEG), similar to measurements using the CPC capacitance sensor.

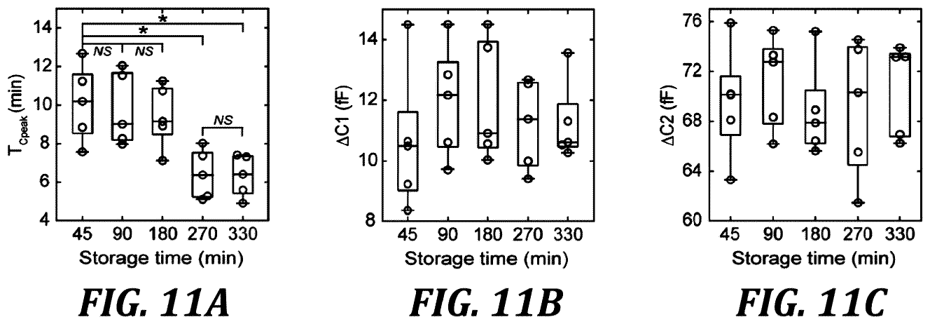

To ensure excellent data repeatability and reliability, the sample size for the sensing parameters characterization study as shown in FIG. 3A-3E was predetermined to be five healthy donors to account for biological variability with two replicate measurements each. For TEG comparison study, blood samples from twelve healthy donors with single replicate measurement was used. Three hemophilia patient samples and simulated thrombocytopenia samples were used to investigate the potential clinical applications for the sensor. Platelet count and hematocrit of blood samples were obtained as an average of three measurements using a hematology analyzer. The vast majority of the data distributions were found to be normal (Shapiro-Wilk Goodness of Fit for normal distribution, p>0.077) and were analyzed using parametric methods for statistical analysis. For the two measurements that contained distributions significantly skewed from normal, shown herein in FIG. 5F and FIG. 11B, non-parametric methods were used for statistical analysis. Pearson's correlation coefficient (r) was used to evaluate linearity between the pairs of data. One-way analysis of variance (ANOVA) with Tukey's post-hoc or Kruskal-Wallis tests was used to compare the results with three or more groups. Two-sided unpaired t-test was used for comparison between healthy donors and hemophilia patients. Two-sided paired t-test or Wilcoxon matched-pairs signed rank test was used to compare between healthy and thrombocytopenia samples. * denotes a p-value less than 0.05 and was considered statistically significant. ** denotes a p-value less than 0.01 and NS denotes not significant.

A blood sample was placed on a carbon nanotube-paper composite (CPC) capacitance sensor made of multiwall carbon nanotubes (MWCNTs) and cellulose fibers as shown in FIGS. 1A and 1B. The CPC sensor contained a fractured region where MWCNTs-coated cellulose fibers formed numerous conductive cantilevers (FIG. 1b-Crack region, 4×) exhibiting a high fringing field for capacitance measurement. A differential measurement using two CPC capacitance sensors was chosen to minimize the environmental disturbances as shown in FIGS. 1C and 1D. This design provided a capacitance signal with low noise in the order of 200 aF (peak-to-peak, as shown in FIG. 7A). Calibration experiment using reference liquid samples showed that a linear relationship exists between the capacitance change ((C) and the permittivity the references (r=0.99, p<0.00001, n=7; Pearson's correlation, as shown in FIG. 7C). Additional measurements using NaCl solutions confirmed that δC sensitively followed the permittivity decrease from increasing molar concentration of NaCl in DI water (F(2.02,8.07)=414.4, p<0.00001, n=11; ANOVA as shown in FIG. 7C).

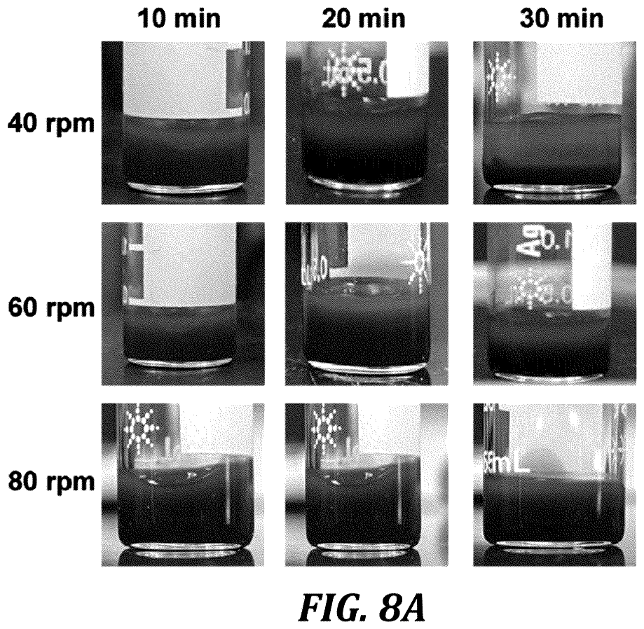

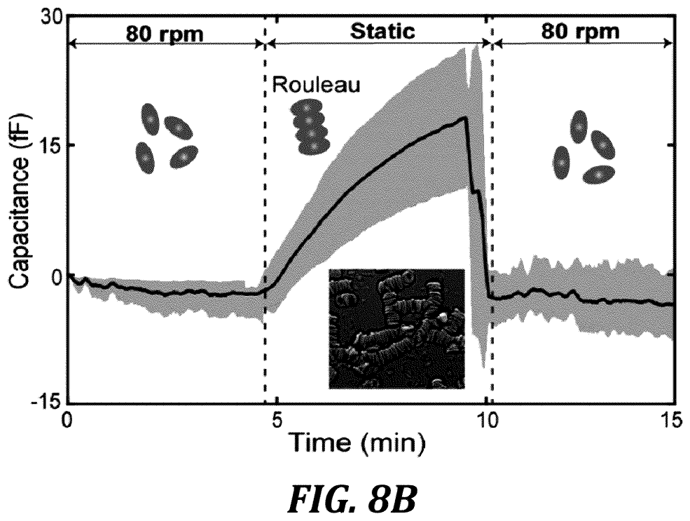

To minimize the differential capacitance variation due to rouleau formation and cell sedimentation, blood samples were gently agitated on an orbital shaker as shown in FIG. 1C. In practice, 80-rpm orbital shaking was applied, since this speed sufficiently minimized cell sedimentation from rouleau formation (as shown in FIG. 8A) and provided a stable reference capacitance (80-rpm cycle, as shown in FIG. 8B). Capacitance measurement at various operating frequency (120 k, 550 k, 1.3M, 3.1M, and 5.3M Hz), using blood samples with a predefined hematocrit (0, 10, 20, 30%), showed that δC at 1.3 MHz was by approximately 125% higher than at 120 kHz for 30% hematocrit as shown in FIG. 2. This finding suggests that the frequency-dependent permittivity response of blood was pronounced at 1.3 MHz, similar to previous studies (Abdalla, 2011; Maji et al., 2017). At this frequency, δC also linearly increased with higher hematocrit (r=0.92, p<0.001, n=3; Pearson's correlation).

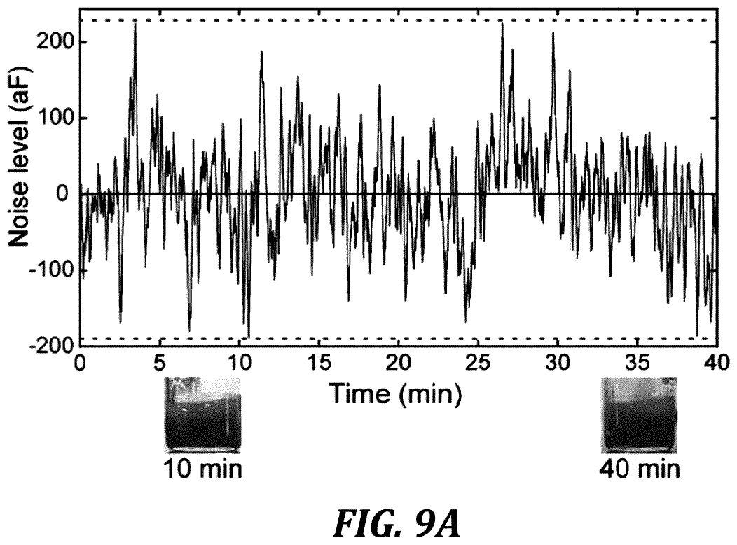

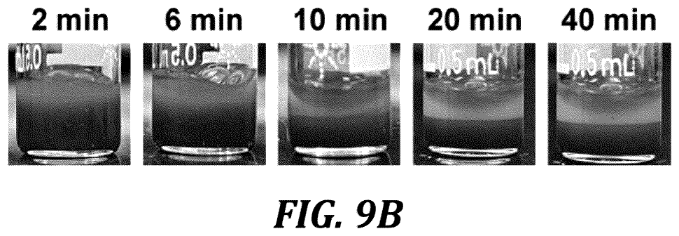

When testing a blood sample with CaCl₂) (final concentration: 12.5 mM), it was observed that capacitance increased and reached a peak value before decreasing to a steady state value in FIG. 3A. To ensure that the signal was from clotting, the same blood sample was tested without recalcification following the same procedure. Capacitance was stable for the inactivated sample with a peak-to-peak change of 414 aF (as shown in FIG. 9A), which confirmed that the capacitance signals in FIG. 3A was caused by clotting. The capacitance trend during clotting was similar to the permittivity changes that have been observed in previous impedance systems. Clotting was not observed in the inactivated sample, which indicated that shaking did not generate a non-physiologically high shear environment to activate the platelets (as shown in FIG. 9A). Moreover, the shaking also did not interfere with the natural progression of clotting for activated blood sample (as shown in FIG. 9B).

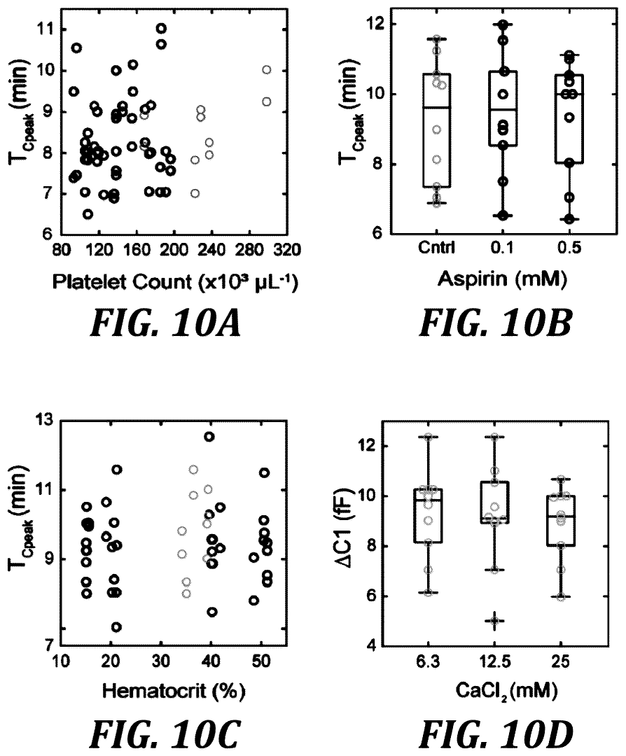

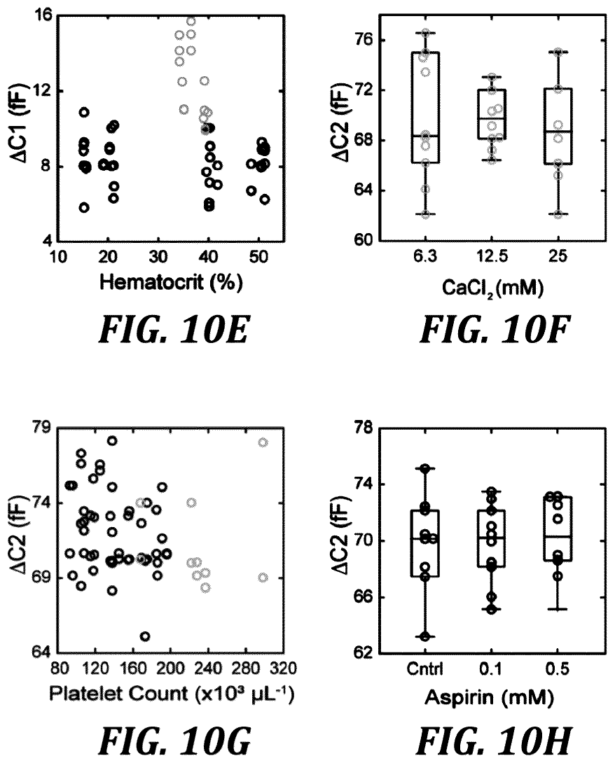

Additionally, the sensing parameters, T_{Cpeak}, ΔC1, and ΔC2, showed distinctive sensitivities towards coagulation function, platelet count or function and hematocrit, respectively. T_{Cpeak} did not change significantly with platelet count (r=0.18, p=0.17; Pearson's correlation, as shown in FIG. 10A), aspirin concentration (F(2,27)=0.082, p=0.92; ANOVA, as shown in FIG. 10B), and hematocrit (r=0.08, p=0.57; Pearson's correlation, as shown in FIG. 10C). ΔC1 did not show a statistically significant difference with CaCl₂) concentration (F(2,27)=0.14, p=0.87; ANOVA, as shown in FIG. TOD) and hematocrit (r=0.037, p=0.80, Pearson's correlation, as shown in FIG. TOE). It was observed that ΔC1 was higher for unmodulated blood samples compared to blood samples modulated to a predefined hematocrit (p<0.0001; Paired t) due to variations in platelet count between the two groups (Grey circles: 249±37, Black circles: 165±27, ×10³ platelets μl⁻¹). This finding was consistent with the result in FIG. 3C, suggesting platelet count to be a major factor affecting ΔC1. ΔC2 had a positive correlation only with hematocrit and no statistically significant changes was detected with variations in CaCl₂) concentration (F(2,27)=0.064, p=0.94; ANOVA, as shown in FIG. 10F), platelet count (r=−0.19, p=0.15; Pearson's correlation, as shown in FIG. TOG) and aspirin concentration (F(2,27)=0.23, p=0.8, ANOVA, as shown in FIG. 5H).

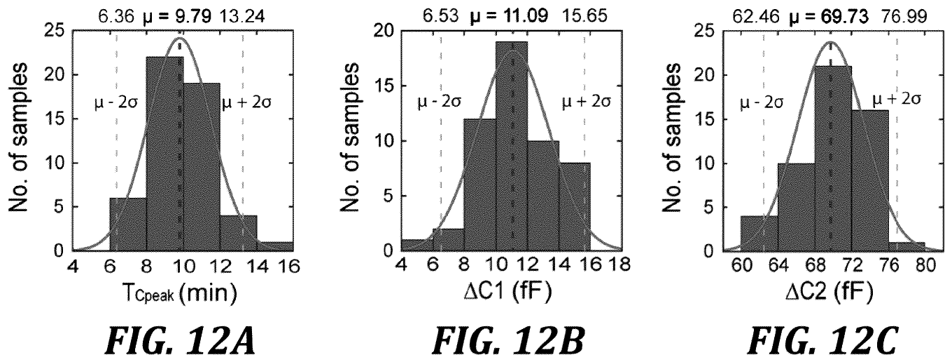

To evaluate the stability of our system, repeated measurements were conducted using room temperature stored blood samples with predefined storage durations (45, 90, 180, 270 and 330 min) after blood draw. A significant decrease in T_{Cpeak} was observed for blood samples stored for 270 and 330 min, relative to measurements at 45 min (F(4,20)=6.72, p<0.045; ANOVA Tukey's post-hoc, as shown in FIG. 11A). No significant difference was observed for blood samples tested at 90 and 180 min compared with 45 min (p>0.96; ANOVA Tukey's post-hoc, as shown in FIG. 11B). No significant difference was observed in ΔC1 (H(4)=0.89, p=0.93; Kruskal-Wallis test) and ΔC2 (F(4,20)=0.28, p=0.89; ANOVA, as shown in FIG. 6C) for all groups. These results have shown good repeatability in the newly introduced sensing parameters for in vitro evaluation of hemostasis using citrated blood. Furthermore, all sensing parameters, T_{Cpeak}, ΔC1, and ΔC2, demonstrated a gaussian distribution trend for the healthy volunteers tested, as shown in FIGS. 12A-12C (p>0.16, n=26; Shapiro-Wilk test).

In some embodiments, the system is configured to evaluate and monitor the effects of other biological impact on hemostasis, clotting, uremia, Von Willebrand disease, and other illnesses known and unknown. To evaluate the potential clinical utility of the sensor, the coagulation time and platelet parameters were compared with the clinically relevant diagnostic parameters of citrate native thromboelastography (CN-TEG) assay.

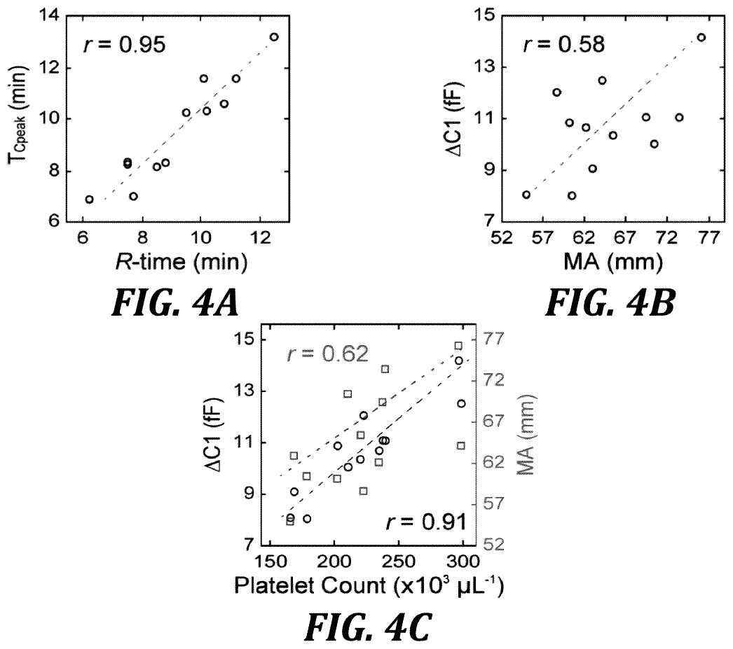

FIGS. 4A-C are graphs of comparison with Thromboelastography (TEG), in accordance with the present technology. FIG. 4A is a graph showing that T_{Cpeak} has a strong positive correlation with the Reaction time (R-time) parameter demonstrating a good agreement with TEG in evaluating coagulation function, where Pearson's r=0.95. FIG. 4B demonstrates a smaller degree of correlation between ΔC1 and Maximum Amplitude (MA) parameter of TEG, with a Pearson's r=0.58. FIG. 4C demonstrates a stronger positive correlation between ΔC1 and platelet count in comparison to MA, where Pearson's r−MA: r=0.62, ΔC1: r=0.91. For all graphs: n=12 healthy donors with single replicate measurement using the presented CPC capacitance sensor and concurrent Citrate Native TEG.

Results illustrated a strong positive correlation between T_{Cpeak} and Reaction time (R-time) as shown in FIG. 4A. The obtained data validated the effectiveness of the presented sensor in evaluating coagulation function and is in good agreement with the viscoelastic TEG assay. However, there was a relatively less degree of correlation between ΔC1 and Maximum Amplitude (MA) as shown in FIG. 4B. The reason for such discrepancy is likely because the MA parameter, which measures clot strength, may be affected by both the number of cells in the clot and passive fibrin elasticity. To examine the hypothesis, the data was rearranged as illustrated in FIG. 4C. Interestingly, a much stronger correlation between ΔC1 and platelet count than to MA was observed (ΔC1: r=0.91, p<0.0001; MA: r=0.62, p=0.033; Pearson's correlation), suggesting ΔC1 may be solely dependent on the platelet count. This also implies that ΔC1 might be a better alternative in evaluating platelet count-related diseases as it may have a higher sensitivity and specificity to platelet count. Furthermore, the common clinical practice to assess the individual contribution of fibrin(ogen) and platelets to MA, is to use a modified TEG assay, where an α_{IIb}β₃ antagonist is introduced to ensure all viscoelastic parameters measured are the result of fibrin formation alone. The results suggest that the proposed sensor could potentially eliminate the need of using additional specialized inhibitors to assay platelets in whole blood, which could further minimize the chances of pre-analytical errors that complicate existing assays.

In some embodiments, the system is configured to evaluate and monitor the effects of other biological impact on hemostasis, clotting, uremia, or Von Willebrand disease, known and unknown conditions, and known and unknown diseases. To examine the usefulness of the sensor in the context of hematologically altered conditions, measurements were conducted using blood samples with clotting disorders. To investigate the clinical relevance in the context of a coagulation disorder, blood samples from three hemophilia patients (2 Hemophilia A, 1 Hemophilia B) were tested.

FIGS. 5A-5F are graphs showing multiparameter hemostasis assessments for hemophilia and thrombocytopenia conditions, in accordance with the present technology. FIG. 5A shows that T_{Cpeak} is prolonged in hemophilia patients indicating the presence of coagulation factor deficiency. In FIGS. 5B and 5C, respectively, ΔC1 and ΔC2 showed no significant difference between healthy and hemophilia patients. FIG. 5D shows T_{Cpeak} is prolonged for blood samples with a thrombocytopenia condition. FIG. 5E demonstrates that ΔC1 is significantly reduced from a severe quantitative platelet depletion. Finally, FIG. 5F shows ΔC2 showed no significant difference between healthy and severe thrombocytopenia conditions. For FIG. 5A-5C, n=26 healthy donors and n=3 hemophilia patients (2 hemophilia A, 1 hemophilia B) with 2 replicate measurements. Additionally, p-values were calculated from two-sided unpaired t-test. For FIGS. 5D-5F, n=3 healthy and thrombocytopenia samples with 2 replicate measurements. For FIGS. 5D and 5E, p-values were calculated from two-sided paired t-test. For FIG. 5F p-value was calculated from Wilcoxon matched-pairs signed rank test. * p<0.05, ** p<0.01 and NS denotes not significant. All box plots with whiskers represent data distribution (Maximum, Third quartile, Median, First quartile, Minimum, with outliers beyond).

The absence of functional factor VIII or factor IX resulted in a significantly higher T_{Cpeak} relative to healthy samples (t=8.47, df=5.5, p<0.001; Unpaired t) as shown in FIG. 5A. This finding showed that T_{Cpeak} was sensitive to coagulation factor deficiency and can potentially be used to monitor coagulation function in hemophilia patients. It was noted that the platelet count and hematocrit were in the normal or close to normal ranges for the hemophilia patient samples (Platelet count: 184±37 platelets μl⁻¹, Hematocrit: 35.8±2.77%; n=3). There was no significant differences in both ΔC1 (t=1.11, df=5.56, p=0.31; Unpaired t) as shown in FIG. 5B and ΔC2 (t=0.66, df=5.85, p=0.54; Unpaired t) as shown in FIG. 5C for hemophilia patients compared to healthy individuals. These results suggested that platelet function or count, and hematocrit were not significantly impacted by a coagulation factor deficiency in these patient samples.

To demonstrate the clinical relevance in the context of a quantitative platelet disorder, platelet counts in healthy blood samples were modulated to be less than 50×10³ platelets μl⁻¹ to induce a thrombocytopenia condition. Interestingly, it was observed that there was a statistically significant increase in T_{Cpeak} for blood samples with a very low platelet count compared to healthy samples (t=3.23, df=5, p=0.023; Paired t) as shown in FIG. 5D. This was in contrast to previous results, where T_{Cpeak} demonstrated characteristic sensitivity to coagulation function by showing no significant difference with variations in platelet count in the range of 93 to 298×10³ platelets μl⁻¹ as shown in FIG. 10A. The reduced T_{Cpeak} observed in blood samples with platelet count less than 50×10³ platelets μl⁻¹ is indicative of the multifaceted role played by platelets in hemostasis. In addition to their key role in primary hemostasis, platelets also play an active role in secondary hemostasis (coagulation cascade) by providing binding sites for coagulation factors in thrombin generation. Therefore, it is possible that a lower activated platelet population could diminish the interaction and localization of the coagulation factors, which further slows down the thrombin generation. As expected, it was observed that there was a significant decrease in ΔC1 compared to healthy samples (t=8.79, df=5, p<0.001; Paired t) as shown in FIG. 5E, reaffirming the sensitivity of ΔC1 to platelet counts. Since hematocrit of thrombocytopenia samples was close to healthy samples (Thrombocytopenia: 40.7±2.9%; Healthy: 38.1±2.1, n=3), no statistically significant difference was observed in ΔC2 (W=9, p=0.44; Wilcoxon matched-pairs signed rank test) as shown in FIG. 5F.

Taken together, these studies demonstrated the potential usefulness of our sensor and highlight the advantages of a simultaneous assessment of cellular and non-cellular components of hemostasis. Furthermore, these preliminary results suggest that the sensor can be used to study the synergistic relationship between coagulation factors, platelets and erythrocytes in the hemostatic process for normal and different disease states.

Motivated by the importance of having a rapid, accurate and convenient hemostasis assessment device, the first reported capacitance-based approach with a unique ability to multiplex the assessment of coagulation function, platelets and hematocrit in a single measurement is described herein. The CPC capacitance sensor described here provides several attractive advantages over existing clinical assays and devices as summarized in Table 1. Its ease of use without the necessity of extensive sample preparation or to be performed only by a highly trained laboratory personnel, and additional benefits, such as non-contact measurement, high reusability and low cost, makes it an ideal tool to readily evaluate clotting status while significantly reducing the financial burden on primary care resources. It may also be used for therapeutic monitoring of anticoagulants, antiplatelet agents, and factor replacement.

| | Presented | | Tradi- | Blood |
|---|---|---|---|---|
| | CPC | | tional | gas or |
| | capaci- | Visco- | labo- | hand- |
| | tance | elastic | ratory | held |
| Features | Sensor | Assays | assays | analyzers |
| Provide global assessment | ✓ | | | |
| of hemostasis and | | | | |
| evaluate bleeding | | | | |
| risk in a single device | | | | |
| Provide global assessment | ✓ | ✓ | | |
| of hemostatic status | | | | |
| (coagulation time + | | | | |
| platelet function) | | | | |
| Can measure platelet count | ✓ | | ✓ | |
| Can measure hematocrit | ✓ | | ✓ | ✓ |
| Rapid Turnround Time | ✓ | ✓ | | ✓ |
| (TAT): No need for | | | | |
| blood sample processing | | | | |
| Ease of use: No need for | ✓ | | | ✓ |
| trained laboratory staff | | | | |
| Cost efficient: Reusability | ✓ | | | ✓ |

Table 1 illustrates advantages of the presented CPC capacitance sensor over currently available alternate approaches. The principle of blood permittivity variation during clotting has been described previously. Earlier attempts using impedance-based sensors have demonstrated the clinical utility by differentiating limited aspects of coagulation and platelet function between normal and pathophysiologic states. However, the presented capacitance-based approach also offers a simultaneous hematocrit assessment not found in any existing clinical devices including the previous impedance-based sensors.

Simultaneous assessment of clotting function and hematocrit can facilitate the development of automated alert or correction algorithm for blood samples with elevated hematocrit (>60%), and eliminate the need for in vitro citrate adjustment. Fibrinogen (Factor I), a key clotting parameter converted to fibrin by thrombin during coagulation, plays a key role in the rapid encapsulation of blood cells and in the propagation of platelet contractile forces through the mesh network for clot retraction. Reduced and/or dysfunctional fibrinogen can lead to pathological bleeding. In addition, various blood physiological parameters such as blood types, concentration of ions (such as Na⁺, Ca²⁺ and K⁺), proteins (such as albumin) and vitamins (such as Vitamin K) may have an impact on blood hemostatic ability and could also influence the inferences from clotting assays. It remains to be seen whether the CPC capacitance sensor could offer reliable hemostatic assessments with the presence of these biological variabilities.

Another unique feature of the sensor is the ability to provide diagnostic parameters with distinctive sensitivity to coagulation function, platelet count or function, and hematocrit. Specifically, in the example system the coagulation and platelet function assessments were not interfered by variations in hematocrit (as shown in FIGS. 10C and 10E). This was in contrast to viscoelastic whole blood assays, which can provide inaccurate assessments for abnormal hematocrit settings. ΔC2 provided an exclusive assessment of hematocrit by not changing significantly with variations in clotting activation levels, platelet count and platelet function (as shown in FIG. 10F-10H). The blood permittivity response at 1.3 MHz was from the accumulation of charge carriers at the interface between the erythrocyte membrane and its cytoplasm. The experimental results suggest that the magnitude of capacitance change (o permittivity change) due to redistribution of these accumulated charge carriers from cellular aggregation and encapsulation of the aggregate structures in a fibrin mesh during the clot growth phase, was dependent on the erythrocyte concentration in whole blood.

The results suggest that the presented sensor affords a simple approach for studying the synergistic relationship between cellular and non-cellular components of hemostasis in normal and different disease states. For example, platelet function in hemophilia patients has been debated and few studies have reported various alterations, such as increased platelet P-selectin expression, lower aggregation upon co-incubation with tissue factor, or reduced platelet contractile forces during clot formation in comparison to healthy individuals. However, these conclusions were from assays on platelet suspensions or platelet-rich plasma which only partially reflect platelet function in hemostasis in vivo. Described herein are results from a limited number of hemophilia patients suggesting that platelet function was not significantly impacted by hemophilia conditions as shown in FIG. 5B. Data obtained from a small number of thrombocytopenia samples revealed a prolonged T_{Cpeak} (i.e., coagulation time) as shown in FIG. 5D. Similar observations have been made from previous assays, however, these existing assays cannot interpret such prolongation in coagulation time as arising from a severe platelet depletion without an independent platelet count measurement. In the disclosed device, in addition to a prolonged T_{Cpeak}, a significant decrease in ΔC1 was observed, as it primarily depends on platelet count, therefore the prolongation in coagulation time may be better deciphered as originating from the reduced platelet count. Additionally, it is also possible to integrate an array of CPC capacitance sensors to conduct parallel assays to analyze blood samples with complex or multiple clotting disorders. These highlight the research-enabling aspect of our technology and the ability to test or create new hypotheses. In a clinical setting, parallel clotting assays could potentially expedite the assessment of bleeding risks in patients, who are not easily diagnosed from a single assay.

In some embodiments, to circumvent the reference signal changes due to rouleau formation and subsequent sedimentation, the system requires constant mechanical vibration/shaking, which limits the realization as a handheld analyzer like CoaguChek (Roche Diagnostics), Xprecia Stride (Siemens Healthineers), or i-STAT (Abbott). While these handheld analyzers may offer a more convenient way to extract physiological properties of blood at bed-side, they have shown to exhibit variable performance and are limited to specific applications like monitoring patients on warfarin therapy. Furthermore, they don't provide information on platelet function or count, resulting in a crude snapshot of the hemostatic status. The majority of the clotting assays are still limited to sophisticated laboratories with well-trained operators and personnel for interpreting the results. The long delay associated with these assays limits their use in acute care settings or patient with active bleeding. In comparison, the system described herein can easily be realized as an automated device with minimal human interventions because of there is no need for sample processing, and there are limited disposables and minimal steps involved for a clotting measurement. In some embodiments, the system components including the orbital shaker can be customized to compact sizes using rapid prototyping techniques. Additionally, the instrumentation for capacitance sensor can also be miniaturized on custom-made circuit board. In some embodiments, the system can be an automated compact self-contained bench top version of the presented system.

FIG. 6 is a SEM image of an example Carbon nanotube composite, in accordance with the present technology. The images of the CPC surface shows that the cellulose fibers are uniformly coated with MWCNTs.

FIGS. 7A-7D are example capacitance sensor noise level, calibration, and characterization in accordance with the present technology. FIG. 7A shows a representative sensor noise level for DI water as detector and reference sample at 80-rpm orbital shaking; peak-to-peak noise, 205 aF; and RMS noise, 34 aF. On the horizontal axis is time in minutes. On the vertical axis is noise level in aF. FIG. 7B is a graph showing the protocol for sensor calibration, characterization and operating frequency study. On the horizontal axis is time in minutes, and on the vertical axis is capacitance in Ff. FIG. 7C is a graph of the relative permittivity of the sample liquid in comparison to other standards. On the horizontal axis is relative permittivity (εᵣ) and on the vertical axis is δC in fF. δC linearly increases with the increasing relative permittivity (εᵣ) of the sample liquid. For comparison, IPA, Acetone, Ethanol, 40% Ethanol, 10% IPA, 2.5% IPA, and DI water were also plotted. Pearson's r=0.99, and the average slope was 2.22 fF εᵣ⁻¹. FIG. 7D is a graph of molar NaCl concentrations. On the horizontal axis is NaCl concentration, and on the vertical axis is δC in fF. δC exponentially decreases with increasing molar concentrations of NaCl (0, 0.05, 0.1, 0.15, 0.2, 0.5, 1, 2, 3, 4 and 5 M). The goodness of fit, R2=0.89. The permittivity of NaCl solutions exponentially decreases with increasing molar concentrations of NaCl and δC sensitively followed this decrement. The inset shows that the decrement was linear for dilute solutions (≤0.5 M) with a Pearson's r=0.97. In both FIGS. 7C and 7D, the circle represents mean from 5 replicate measurements, and error bar is a standard deviation.

FIG. 8A is an example observation of sedimentation at different orbital rotational speed, in accordance with the present technology. Shown is a visual observation of erythrocyte rouleau sedimentation at different orbital rotational speed (rpm). Orbital shaking at 80-rpm sufficiently minimized sedimentation and maintained homogeneity in the citrated blood sample.

FIG. 8B is a graph of the time course variation of capacitance for healthy whole blood subjected to orbital shaking, in accordance with the present technology. On the horizontal axis is time in minutes, and on the vertical axis is capacitance in fF. Shown is the time course variation of capacitance for healthy citrated whole blood subjected to repetitive cycle of orbital shaking and static. Capacitance increases with erythrocyte rouleau formation during the static cycle; n=3. The shaded region denotes standard deviation, and the dotted lines indicate timepoint for switching cycles.

FIG. 9A is a graph of a capacitance signal after orbital shaking, in accordance with the present technology. On the horizontal axis is time in minutes. On the vertical axis is the noise level in aF. Along the horizontal axis is two representative blood samples at 10 and 40 minutes. Shown is the stability of the capacitance signal at 80-rpm orbital shaking for non-activated blood sample; peak-to-peak noise, 414 aF and RMS noise, 68 aF. No visual clot formation was observed in the citrated blood sample photographed at 10 min and 40 min of orbital shaking.

FIG. 9B is example clotting after orbital shaking, in accordance with the present technology. Shown are representative blood samples at 2, 6, 10, 20, and 40 minutes. Orbital shaking at 80-rpm did not interfere with the natural progression of clotting when the detector blood sample was activated using 12.5 mM of CaCl₂).

FIGS. 10A-10H are graphs of sensing parameters, in accordance with the present technology. FIGS. 10A-10C show the independence of T_{Cpeak} with (a) Platelet count in the range 93-298×10³ platelets μL-1, (b) Aspirin treatment, and (c) Hematocrit in the range 15.1%-51.2%, respectively. FIGS. 10D-10E show the independence of ΔC1 with respect to activating CaCl₂) concentration and hematocrit in the range 15.1%-51.2% respectively. Unmodulated raw samples (gray circles) had higher platelet counts (Average: 249±37×10³ platelets μl⁻¹) compared to modulated samples (black circles; Average: 165±17×10³ platelets μl⁻¹), which shows increased ΔC1 values. FIGS. 10F-10H show the independence of ΔC2 with activating CaCl₂) concentration, platelet count in the range 93-298×10³ platelets μl⁻¹, and, aspirin treatment respectively. For all graphs in FIGS. 10F-10H n=5 healthy donors with two replicate measurements. Black circles are results from blood samples treated in vitro to modulate platelet count or platelet function or hematocrit, and gray circles are results from blood samples without any such treatments. All box plots with whiskers represent data distribution (Maximum, Third quartile, Median, First quartile, Minimum). No statistically significant difference between groups or correlation between pairs of data was found from one way ANOVA and Pearson's r respectively.

FIGS. 11A-11C are graphs of stability assessments by an example sensor in accordance with the present technology. On the horizontal axis is storage time in minutes. On the vertical axis is T_{Cpeak} ΔC1, and ΔC2, respectively. FIG. 11A shows T_{Cpeak} drops for whole blood tested after 270 min from blood draw, indicating a trend towards hypercoagulability. FIG. 11B and 11C show that ΔC1, and ΔC2 remained stable up-till 330 min. For all graphs in FIG. 11A-11C, n=5 healthy donors. All box plots with whiskers represent data distribution (Maximum, Third quartile, Median, First quartile, Minimum). For (a): p-values were calculated from one way ANOVA with Tukey's post-hoc. * p<0.05, ** p<0.01 and NS denotes not significant. No statistically significant difference between groups was found from Kruskal-Wallis test as used in FIG. 11B and one way ANOVA as used in FIG. 11C.

FIGS. 12A-12C are graphs of example distribution sensing parameter for health volunteers in accordance with the present technology. On the vertical axis of FIGS. 12A-12C is the number of samples. On the horizontal axis is the T_{Cpeak}, ΔC1, and ΔC2 respectively. The normal distribution of T_{Cpeak} (p=0.16), ΔC1 (p=0.72) and ΔC2 (p=0.66)) for 26 healthy samples with two replicate measurements. p-values were calculated from Shapiro-Wilk Goodness of Fit for normal distribution (p>0.05 is considered normally distributed).

Described herein is a non-contact capacitance-based hemostasis assessment using a novel carbon nanotube-paper composite (CPC) sensor. CPC capacitance measurements at 1.3 MHz provided three sensing parameters, namely T_{Cpeak}, ΔCT and ΔC2, to independently assess coagulation function, platelet function or count, and hematocrit respectively. The presented sensor was characterized using reference permittivity liquids and then applied to evaluate the hemostatic ability of blood samples with varying clotting activation levels, quantitative and qualitative platelet defects, and different hematocrits. The testing results showed good agreements with a conventional TEG analyzer. Potential clinical usefulness of the sensor was demonstrated by testing hemophilia patient samples and blood samples simulated with thrombocytopenia condition. Finally, the presented CPC capacitance sensor is a promising new diagnostic device for convenient comprehensive evaluation of hemostasis with attractive advantages such as whole-blood based non-contact evaluation of multiple key clotting biomarkers with high accuracy, high sensitivity and low cost.

1. A capacitive sensor system configured to measure capacitance, the system comprising:
a sample volume;
a sample capacitive sensor configured to measure the capacitance of the sample volume without physical contact between the sample capacitive sensor and the sample volume;
a control capacitive sensor;
a differential sensing subsystem configured to measure a control sensor volume using the control capacitive sensor; and
electrical circuitry connected to both the control capacitive sensor and the sample capacitive sensor.

2. The capacitive sensor system of claim 1, wherein the sample capacitive sensor is a carbon-nanotube cellulose paper composite sensor

3. The capacitive sensor system of claim 1, wherein the control sensor is a carbon-nanotube cellulose paper composite sensor.

4. The capacitive sensor system of claim 1, wherein the electrical circuitry for differential capacitive measurement uses pulse-counting or resonance between 10 kHz to 10 MHz.

5. The capacitive sensor system of claim 1, wherein the electrical circuitry for differential capacitive measurement is configured to measure the sample capacitive sensor capacitance from the change in resonant frequency of an inbuilt tank circuit, in parallel to the sample capacitive sensor.

6. The capacitive sensor system of claim 1, wherein the electrical circuitry is configured to calibrate temperature change.

7. (canceled)

8. The capacitive sensor system of claim 1, wherein the control volume is used to calibrate the capacitive sensor system related to a characteristic selected from volume, dielectric constant, liquid temperature, or combinations thereof.

9. The capacitive sensor system of claim 1, further comprising:
a sample vial configured to contain the sample volume; and
a control vial configured to contain the control volume.

10. The capacitive sensor system of claim 1, further comprising one or more housings configured to position the sample capacitive sensor in relation to the sample volume and position the control capacitive sensor in relation to the control volume.

11. The capacitive sensor system of claim 1, wherein the system further comprises an orbital oscillator or shaker mechanism placed beneath the sample capacitive sensor and the control capacitive sensor.

12. (canceled)

13. The capacitive sensor system of claim 1, wherein the sample volume is whole blood.

14.-16. (canceled)

17. The capacitive sensor of claim 13, wherein the system is configured to detect both quantitative platelet defects and qualitative platelet defects.

18. The capacitive sensor of claim 13, wherein the system is configured to measure and count platelets and hematocrit.

19. The capacitive sensor system of claim 13, wherein the system is configured to evaluate or monitor the effects of antiplatelet factors and anticoagulant factors on hemostasis.

20.-21. (canceled)

22. The capacitive sensor system of claim 13, wherein the system is configured to evaluate and monitor the effects of other biological impact on hemostasis, clotting, uremia, or Von Willebrand disease.

23. The capacitive sensor system of claim 1, wherein the sample volume is not within a plane formed by electrodes of the sample capacitive sensor.

24. The capacitive sensor system of claim 23, wherein the sample volume, at its nearest point, is set apart from the plane formed by the electrodes by a distance of 0.1 mm-10 mm.

25. A method of assessing one or more blood conditions using the sensor of any of claim 1, the method comprising:
filling a sample vial and a control vial with an agonist solution to activate clot formation;
placing a whole blood sample into the sample vial;
placing a control sample into the control vial;
measuring the capacitance of the whole blood sample with a sample capacitance sensor that is not in contact with the whole blood sample using a differential capacitance measurement; and
concurrently measuring the clotting ability, hematocrit, and platelet count of the whole blood sample.

26. The method of claim 25, wherein the whole blood sample is between 1 and 500 ml

27. The method of claim 25, wherein the whole blood sample is a pinprick.

28.-29. (canceled)
